(19) United States
(12) Patent Application Publication
(10) Pub. No.: US 2025/0095868 A1
(43) Pub. Date: Mar. 20, 2025

(54) FORPARTUM ALL IN ONE POSTPARTUM WELLNESS APP

(71) Applicant: Jamilia Shani Jackson-Beasley, Tucson, AZ (US)

(72) Inventor: Jamilia Shani Jackson-Beasley, Tucson, AZ (US)

(21) Appl. No.: 18/368,887

(22) Filed: Sep. 15, 2023

(51) Int. Cl. G16H 80/00; G16H 20/60

(52) U.S. Cl. CPC G16H 80/00; G16H 20/60

(57) ABSTRACT

The “Forpartum All in One Postpartum Wellness App” is a pioneering postpartum support solution that responds to the pressing need for comprehensive care for new mothers and parents during the postnatal period. This innovative app offers a holistic approach to postpartum wellness, encompassing a 52-week wellness tracker, assessments, thought-provoking journal prompts, purposeful exercise plans, personalized meal planning, immediate access to a postpartum hotline, interactive live support sessions, and a direct conduit to a network of seasoned professionals. With a focus on nurturing well-being across physical, emotional, and nutritional dimensions, this app fills a critical gap in postnatal care, empowering individuals to embark on their postpartum journey with resilience, confidence, and the support of a caring community.

### FIELD OF INVENTION

The field of “Postpartum Wellness and Support” involves a multifaceted approach to improving the well-being of individuals during the postpartum period, with a focus on addressing physical, emotional, and nutritional needs through various interventions and support mechanisms. The “Forpartum All in one Postpartum Wellness App” is a specific example of an innovation within this field, providing accessible and comprehensive postpartum support through digital technology.

### BACKGROUND OF THE INVENTION

The postpartum period, commonly referred to as the “fourth trimester,” represents a pivotal and transformative time in the lives of new mothers and parents. It is a period marked by both joy and profound adjustment as individuals navigate the challenges and responsibilities of caring for a newborn child while also focusing on their own recovery and well-being.

During this crucial phase, new mothers and parents often encounter a multitude of physical, emotional, and practical challenges. The demands of newborn care, coupled with the physical toll of childbirth, can leave individuals feeling exhausted, overwhelmed, and in need of comprehensive support. Furthermore, the postpartum period is frequently accompanied by a range of emotional experiences, from the elation of bonding with a new baby to the strains of sleep deprivation and, in some cases, the profound challenges associated with postpartum mental health.

While the importance of postpartum care and support is widely acknowledged, there remains a persistent gap in accessible and comprehensive resources for new mothers and parents. Many existing solutions and support networks are fragmented, lacking in depth, or inaccessible to those in need. Additionally, the unique needs of individuals during the postpartum period often extend beyond traditional healthcare services, encompassing aspects of physical recovery, emotional well-being, and nutritional guidance.

Existing approaches to postnatal care often fall short in addressing the holistic and multifaceted nature of the postpartum experience. They may focus primarily on medical aspects or overlook the critical importance of emotional and mental health support. Consequently, new mothers and parents frequently find themselves without the comprehensive care and guidance required during this significant life transition.

In response to these challenges, the “Forpartum All in one Postpartum Wellness App” has been innovatively designed to bridge the gap in postpartum support and address the holistic needs of individuals during the postnatal period. By offering a wide range of features, including a 52-week tracker, assessments, journal prompts, gentle movement exercises, personalized meal plans, a readily accessible postpartum hotline, live support sessions, and access to expert postpartum professionals, the app aims to provide a comprehensive solution to the challenges faced by new mothers and parents. It seeks to empower individuals with the resources and support necessary to navigate the postpartum journey with confidence, resilience, and well-being.

### SUMMARY OF THE INVENTION

The “Forpartum All in one Postpartum Wellness App” represents a groundbreaking solution for addressing the comprehensive needs of new mothers and parents during the postpartum period, a transformative and often challenging phase in their lives. This innovative app has been meticulously designed to offer a wide array of supportive features and resources that empower individuals to navigate this critical life stage with confidence, health, and well-being.

Key Features of the “Forpartum All in one Postpartum Wellness App” include:
1. 52-Week Tracker: The app provides a dedicated 52-week tracker, offering a structured and accessible way for users to monitor their postpartum journey. This comprehensive tracking system helps individuals keep a record of their physical and emotional progress and milestones.
2. Assessments: Users can access assessments tailored to their specific postpartum needs. These assessments allow individuals to gain insights into their well-being, identify areas that may require attention, and receive personalized recommendations for support.
3. Journal Prompts: The app offers a curated collection of journal prompts designed to encourage reflection and self-expression. Users can use these prompts to document their experiences, thoughts, and emotions during the postpartum period, fostering a sense of self-awareness and emotional well-being.
4. Gentle Movement Exercises: Recognizing the importance of physical recovery and well-being, the app provides gentle movement exercises suitable for postpartum individuals. These exercises promote strength, flexibility, and overall physical health.
5. Personalized Meal Plans: Nutritional guidance is a vital component of postpartum wellness. The app offers personalized meal plans tailored to the unique dietary needs of individuals during the postnatal period, ensuring they receive the nourishment necessary for recovery and vitality.
6. Readily Accessible Postpartum Hotline: The app includes a readily accessible postpartum hotline, providing a lifeline for individuals in need of immediate support, guidance, or assistance. Trained professionals are available to address concerns and provide valuable information.
7. Live Support Sessions: To foster a sense of community and connection, the app hosts live support sessions where users can engage with experts, fellow new parents, and healthcare professionals. These sessions offer a platform for shared experiences, advice, and emotional support.
8. Access to Expert Postpartum Professionals: Users of the app have the unique advantage of accessing a network of expert postpartum professionals, including healthcare providers, counselors, dietitians, and fitness experts. This ensures that individuals receive tailored guidance and support based on their specific needs.

In summary, the “Forpartum All in one Postpartum Wellness App” is a comprehensive and user-centric solution that fills a critical gap in postpartum care and support. It empowers new mothers and parents with the tools, resources, and expertise needed to embark on their postnatal journey with resilience, confidence, and well-being. This innovative app is poised to transform the postpartum experience, offering holistic support that encompasses physical, emotional, and nutritional aspects, ultimately contributing to the health and happiness of new families.

### DETAILED DESCRIPTION OF THE INVENTION

The “Forpartum All in one Postpartum Wellness App” is an innovative and user-centric mobile application specifically designed to provide comprehensive support and guidance to new mothers and parents during the postpartum period. This app is uniquely positioned to address the diverse and evolving needs of individuals in the postnatal phase, offering a wide range of features and functionalities to promote holistic well-being.

Key Features and Functionalities:
1. User-Friendly Interface: The app boasts an intuitive and user-friendly interface that caters to individuals at varying levels of technological familiarity. It ensures easy navigation and accessibility for all users.
2. 52-Week Tracker: At the heart of the app is a 52-week tracker, allowing users to record and monitor their postpartum journey week by week. This feature facilitates goal setting, progress tracking, and the celebration of milestones, fostering a sense of accomplishment and self-awareness.
3. Assessment Tools: The app offers a suite of assessment tools that users can utilize to evaluate their physical, emotional, and nutritional well-being. These assessments are designed to provide personalized insights and recommendations, helping individuals identify areas that may require attention.
4. Journal Prompts: To encourage self-expression and reflection, the app includes a library of thoughtfully crafted journal prompts. Users can use these prompts to document their experiences, thoughts, and feelings during the postpartum period, promoting emotional well-being and self-awareness.
5. Gentle Movement Exercises: Recognizing the importance of physical recovery, the app features a curated collection of gentle movement exercises suitable for postpartum individuals. These exercises promote physical strength, flexibility, and overall wellness.
6. Personalized Meal Plans: Nutritional guidance is a cornerstone of the app. It offers personalized meal plans tailored to the unique dietary needs of individuals during the postnatal period. Users can access recipes, dietary recommendations, and meal planning tools to support their overall health.
7. Readily Accessible Postpartum Hotline: In times of urgent need or when immediate support is required, the app provides a readily accessible postpartum hotline. Trained professionals and counselors are available 24/7 to address concerns, provide guidance, and offer emotional support.
8. Live Support Sessions: The app hosts live support sessions, bringing together experts, fellow new parents, and healthcare professionals in a virtual community. These interactive sessions provide a platform for shared experiences, advice, and emotional support, fostering connections and reducing feelings of isolation.
9. Access to Expert Postpartum Professionals: Users have the valuable opportunity to access a network of expert postpartum professionals. These professionals include healthcare providers, counselors, dietitians, lactation consultants, and fitness experts. Users can schedule virtual consultations and receive personalized guidance and support.
10. Privacy and Security: The app prioritizes user privacy and data security, ensuring that sensitive information is protected and handled with the utmost care. Strict security measures are in place to safeguard user data.

In addition to the previously mentioned features and functionalities, the “Forpartum All in one Postpartum Wellness App” places a strong emphasis on addressing the following six pillars of postpartum care:
1. Nutrition Support: The app offers tailored nutritional guidance and personalized meal plans to ensure that new mothers and parents receive the essential nutrients needed for postnatal recovery and overall well-being.
2. Mental Health: Recognizing the significance of mental health during the postpartum period, the app provides a comprehensive suite of tools and resources to support emotional well-being. This includes access to assessments, therapeutic interventions, and a nurturing community to address postpartum depression, anxiety, and stress.
3. Physical Recovery: The app's gentle movement exercises and exercise plans are designed to facilitate physical recovery after childbirth. These exercises promote strength, flexibility, and the restoration of physical vitality.
4. Nervous System: Acknowledging the intricate connection between the nervous system and postpartum well-being, the app offers strategies and practices to calm and nurture the nervous system, aiding in stress reduction and relaxation.
5. Community Care: Building a sense of community is fundamental to the app's approach. It fosters connections among new parents, creating a supportive network for sharing experiences, advice, and emotional support.
6. Sleep Support: The app provides guidance and strategies to address sleep-related challenges often experienced by new parents. It includes tips for improving sleep quality and creating healthy sleep routines for both parents and newborns.

### INNOVATION AND UNIQUENESS

What sets the “Forpartum All in one Postpartum Wellness App” apart is its comprehensive and user-tailored approach to postpartum care. The app combines tracking, assessment, emotional support, nutrition, and access to expert professionals, all within a single platform. This holistic approach acknowledges that postnatal wellness encompasses physical, emotional, and nutritional aspects, and it empowers users to make informed decisions about their well-being.

In conclusion, the “Forpartum All in one Postpartum Wellness App” represents a pioneering solution to address the multifaceted needs of new mothers and parents during the postpartum period. Its features and functionalities are meticulously designed to empower individuals to navigate this transformative life stage with resilience, confidence, and holistic well-being. This app is poised to make a significant impact by improving the postpartum experience and promoting the health and happiness of new families.

1. A postpartum wellness app, comprising:
A 52-week tracker for new mothers.
Assessment tools.
Journal prompts.
Gentle movement exercises.
Personalized meal plans.
A postpartum hotline.
Live support sessions.
Access to expert postpartum professionals.
Focus on six pillars of postpartum care: Nutrition Support, Mental Health, Physical Recovery, Nervous System, Community Care, and Sleep Support.
Each of the above claims represents a distinct aspect or feature of the invention, providing a clear and concise description of the app's functionalities. These claims serve to define the scope of the invention and what is being sought for protection through the patent application.
